(19) United States
(12) Patent Application Publication
(10) Pub. No.: US 2005/0154032 A1
(43) Pub. Date: Jul. 14, 2005

(54) Methods of using zonisamide as an adjunctive therapy for partial seizures

(75) Inventor: Ivan Lieberburg, Berkeley, CA (US)

Correspondence Address: Finnegan, Henderson, Farabow,;Garrett & Dunner, L.L.P., 1300 I Street, N.W., Washington, DC, 20005-3315, US

(73) Assignee: Eisai Co., Ltd, Tokyo (JP)

(21) Appl. No.: 10/752,516

(22) Filed: Jan. 8, 2004

(51) Int. Cl. A61K 31/42

(52) U.S. Cl. 514/379

(57) ABSTRACT

Methods of using zonisamide as an adjunctive therapy for partial seizures are disclosed. In particular, the methods enhance the safety of patients taking pharmaceutical formulations of zonisamide by providing information that increases the awareness of hyperammonemia as a possible side effect; wherein the patients and/or prescribing physicians and other medical care providers are advised to monitor for hyperammonemia and employ methods that will improve the therapeutic outcome in the few patients who experience hyperammonemia associated with zonisamide therapy.

### FIELD OF THE INVENTION

The present invention generally relates to methods of using zonisamide (3-benzisoxazole methylene sulfonamide) as an adjunctive therapy for partial seizures.

### BACKGROUND OF THE INVENTION

In the United States, over 2 million serious adverse drug reactions (ADRs) occur ever year, with 100,000 associated deaths. This places ADRs as the fourth leading cause of death, ranking ahead of pulmonary disease, diabetes, AIDS, pneumonia, accidents, and automobile deaths. Compounding this problem is the fact that ADRs increase exponentially in patients who take four or more medications concurrently. (See http://www.fda.gov/cder/drug/drugReactions/default.htm, last checked Aug. 20, 2003.)

Most drugs are approved by a Food and Drug Administration review process after an average of 1,500 patient exposures. Clinical trials involving this number of subjects (both healthy volunteers and patients in need of the therapeutic effect of the drug under review) provide a statistically relevant sample of the population from which an assessment of safety and efficacy can be evaluated. However, some drugs have very rare toxicity profiles. Bromfenac, for example, causes hepatotoxicity in 1 out of 20,000 patients. For drugs with rare toxicity, more than 100,000 patients must be exposed to generate a warning signal for the adverse event. In instances where an adverse event is identified in association with a human therapeutic, government regulations require a post-approval follow-up after the drug has been taken to market.

Examples of very serious post-marketing events that have been identified in the recent past include Fen-Phen (fenfluramine-phentermine combination therapy) for weight loss and Rezulin (troglitazone) for diabetes, both of which were later removed from the market because the ADR risks outweighed the therapeutic benefits. Statistical and clinical analysis of large adverse event databases collected by post-marketing surveillance is one method by which identification of the rarer ADRs can be made. For more background on the occurrence and identification of ADRs see, for example, Lazarou, J. et al. JAMA 279(15):1200-1205 (1998), and Gurwitz, J. H. et al. Am J. Med. 109(2):87-94 (2000). For a discussion of techniques and difficulties inherent in identifying ADRs in adjunctive therapies of epileptic seizures, see French, J. Epilepsia 43(9): 951-955 (2002), which is hereby incorporated by reference in its entirety.

While Rezulin and Fen-Phen are notable for their extreme and potentially irreversible nature, other adverse drug reactions can be minimized or more easily reversed if they are recognized early, and appropriate and timely medical intervention is made. A few examples of frequently reversible adverse events are cardiac arrhythmias, liver function abnormalities, and irregularities in hematopoiesis. Thus, there remains a need for methods for identifying, for detecting and for treating adverse events associated with drug therapy, in a timely and informed manner.

### DESCRIPTION OF THE INVENTION

Unexpectedly it has been found by the applicants that zonisamide therapy in a very small percentage of patients (available estimates are about 1:1,222,453 based upon the estimates of U.S. and Japanese exposure) can precipitate hyperammonemia. It also has been found that by curtailing (either by removal or tapering off) the administration of zonisamide dosing, alone or in conjunction with other concomitant medications, alleviation and minimization of this severe adverse event is possible. This is particularly the case when medical intervention to manage the disease and/or removal, reduction, or tapering off of zonisamide is instituted rapidly.

Accordingly, the present invention is directed to methods of using zonisamide for a regulatory agency approved use (e.g., as an adjunctive therapy for partial seizures). The methods improve the safety of zonisamide therapy for patients receiving administrations of the drug, or those who are in need of zonisamide therapy.

In some embodiments, the methods of using zonisamide as an adjunctive therapy for partial seizures improves the safety and health of patients taking zonisamide by increasing the awareness of the patient or patient's guardian that hyperammonemia is a possible side effect. Accordingly, a patient may be provided with a therapeutically effective amount of zonisamide, and the patient or the patient's guardian may be informed that irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing are symptoms of hyperammonemia that require prompt medical evaluation if such symptoms are experienced by the patient. As a result, the patient or patient's guardian can monitor for signs and symptoms of hyperammonemia, and seek medical attention if such symptoms occur in order to obtain appropriate tests, diagnosis, and treatment. In some embodiments, the present methods reduce the risk of hyperammonemia in patients receiving zonisamide therapy.

In other embodiments, the present invention provides methods of using zonisamide as an adjunctive therapy for partial seizures comprising informing a prescribing physician or other medical professional (e.g., an emergency medical worker) that hyperammonemia may result from zonisamide therapy and to monitor a patient who is prescribed zonisamide as an adjunctive therapy for partial seizures for irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing. The prescribing physician or other medical professional also may be advised that when irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing is observed, an appropriate diagnostic be employed to determine whether hyperammonemia is present. In addition, the prescribing physician or other medical professional may be advised to remove, reduce, or taper off the zonisamide dosing in the patient, and initiate appropriate supportive therapy for the underlying condition(s). In this manner, the present methods enable prescribing physicians and other health care professionals to recognize and minimize the risk associated with an adverse event, namely hyperammonemia, which rarely occurs in some patients who receive zonisamide therapy.

The present methods also include methods of administrating zonisamide as an adjunctive therapy for partial seizures comprising providing packaging that includes a pharmaceutical formulation of zonisamide along with information providing a warning that zonisamide may cause hyperammonemia in some patients and that one or more symptoms chosen from the group of irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, and decorticate or decerebrate posturing are potentially signs of hyperammonemia; and providing the packaging to a patient who has been prescribed zonisamide.

The medical information provided in any of the above described methods concerning the signs and symptoms of hyperammonemia may alternatively be provided in layman's terms, so as to be better understood by patients or non-medical professionals. For example, the listed symptoms of hyperammonemia may include disturbances in awareness or mentation, forgetfulness, confusion, obtundation, coma, disturbances in sleep wake-cycle, nausea, vomiting, alterations in personality, mood disturbances, deterioration in self-care or handwriting, and/or daytime somnolence. Those of skill in the medical art are familiar with the various layman's terms that can be used to describe the symptoms of hyperammonemia.

Other advantages and uses of the present invention will become apparent to those skilled in the art in studying this disclosure; therefore this recitation is not intended to limit the scope of the claims attached hereto.

### DESCRIPTION OF THE EMBODIMENTS

Zonisamide is an antiseizure drug, chemically classified as a sulfonamide and unrelated to other antiseizure agents. Antiepileptic drugs are commonly abbreviated as “AEDs.” The active ingredient is zonisamide, 1,2-benzisoxazole-3-methanesulfonamide. Zonisamide was approved in 2000 for the adjunctive treatment, i.e., taken in conjunction with one or more other AED, treatment of epilepsy in the United States. It was first introduced in Japan approximately 12 years ago, where it also has been used as monotherapy, i.e., without other AEDs as concomitant therapeutics. Zonisamide is not known to be a hepatic enzyme inducer and has been administered adjunctively with almost all of the other regulatory-approved AEDs either in the United States or abroad.

The precise mechanism(s) by which zonisamide exerts its anti-seizure effect is unknown. Zonisamide may produce antiseizure effects through action at sodium and calcium channels. In vitro pharmacological studies suggest that zonisamide blocks sodium channels and reduces voltage-dependent, transient inward currents (T-type Ca²⁺ currents), consequently stabilizing neuronal membranes and suppressing neuronal hypersynchronization, thus suppressing hyperexcitablity in epileptic foci. In vitro binding-studies have demonstrated that zonisamide binds to the GABA/benzodiazepine receptor ionophore complex in an allosteric fashion, which does not produce changes in chloride flux. Other in vitro studies have demonstrated that zonisamide (10-30 μg/mL) suppresses synaptically-driven electrical activity without affecting postsynaptic GABA or glutamate responses (cultured mouse spinal cord neurons) or neuronal or glial uptake of [³H]-GABA (rat hippocampal slices). Thus, zonisamide does not appear to potentiate the synaptic activity of GABA. In vivo microdialysis studies demonstrated that zonisamide facilitates both dopaminergic and serotonergic neurotransmission. Zonisamide also has weak carbonic anhydrase inhibiting activity (about 1/50^{th} the inhibition compared to acetazolamide), and this pharmacologic effect is not thought to be a major contributing factor in the anti-seizure activity of zonisamide.

ZONEGRAN® (the human therapeutic pharmaceutical formulation containing zonisamide) is indicated as adjunctive therapy for the treatment of partial seizures in adults and is supplied by prescription in the form of 25, 50, and 100 mg capsules. The capsule may be divided, so as to offer smaller increments in dosage. Recommended dosing is once or twice daily, the recommended daily dose of 100 mg at the initiation of therapy should not be divided. ZONEGRAN® is given orally and can be taken with or without food. While other therapeutic uses of zonisamide have been reported, such as treatment of obesity and eating disorders, treatment of neuropathic pain, prophylaxis of migraine attacks, and treatment of mania, these are not indications approved by the Food and Drug Administration (FDA) in the United States, and so are called “off-label” uses. Off-label uses, which are within the discretion of the prescribing physician to write, are also encompassed in the methods presented herein.

Prescribing physicians are informed in the product insert (which contains prescribing information approved by the FDA) that, because of the long half-life of zonisamide, up to two weeks may be required to achieve steady-state levels upon reaching a stable dose or following dosage adjustment. Although the regimen described below has been shown to be tolerated, the prescriber may wish to prolong the duration of treatment at the lower doses in order to fully assess the effects of zonisamide at steady state, noting that many of the side effects of zonisamide are more frequent at doses of 300 mg per day and above. Although there is some evidence of greater response at doses above 100-200 mg/day, the increase appears small and formal dose-response studies have not been conducted.

The initial dose should be 100 mg daily. After two weeks, the dose may be increased to 200 mg/day for at least two weeks. It can be increased to 300 mg/day and 400 mg/day, with the dose stable for at least two weeks to achieve steady state at each level. Evidence from controlled trials suggests that ZONEGRAN® doses of 100-600 mg/day are effective, but there is no suggestion of increasing response above 400 mg/day.

Adjunctive therapy for partial seizures in adults denotes that these patients are already on other anti-epileptic medications, but that they are continuing to seize at a rate that has been deemed by their treating physician to require additional (add-on) therapy. For a recent review of AEDs currently available to American physicians, their efficacies for particular types of epileptic seizures and associated ADRs, see: Hio Leppik, Epilepsia 42(Suppl.4): 1-6 (2001).

The use of multiple anti-epileptic medications in the adjunctive setting and other drug combinations increases the likelihood of confluent or interactive ADRS, and also may confuse the treating physician as to the causal agent. For instance, when an attending medical professional is presented with a patient taking a combination of medications and manifesting a particular side-effect, it is difficult to diagnose which of the patient's medications (or combination of medications) is responsible for the observed side effect. Typically, the attending physician must consult the medical literature of known adverse events to identify drug(s).that are most likely to cause the observed side-effects. Known adverse events may also be found in the package drug inserts for each drug. The drug(s) having the higher likelihood of causing the observed side-effects are usually reduced or withdrawn first. When such options are exhausted, the patient may have to be systematically withdrawn from the various drugs until the cause is identified. Since zonisamide is typically prescribed as an adjunctive therapy, it presents such complications when side-effects occur.

This situation is further complicated when side-effects occur that are not normally associated with a particular drug. For example, zonisamide was not previously known to be linked with hyperammonemia in patients receiving ZONEGRAN® therapy; while valproate (see Verrotti, et al., Metabolic Brain Disease, Vol. 17(4), pp. 367-373, 2002) and carbamazepine (see Gentile, et al., Rivista di Neurobiologia, Vol. 39(4), pp. 447-450, 1993) are known to cause hyperammonemia. See also, O'Neil, et al., Postgrad. Med. J., Vol. 78, pp. 316-317, 2002 (noting that more than 30 cases of hyperammonemia have been associated with valproate since 1979). Given this knowledge, a medical professional would not suspect zonisamide to be the likely agent responsible for causing hyperammonemia in a patient exhibiting the relevant symptoms. Consequently, a hyperammonemic patient receiving a combination of these drugs would be withdrawn from the known causative agents, namely, valproate or carbamazepine. Meanwhile, the attending medical professional would have no obvious reason to withdraw such a patient from zonisamide, and would allow the therapy to continue while searching for other causes of the hyperammonemia.

This particular problem involving combination therapies was reported by Tsuchiya, et al. (J. Japan Epilepsy Society, Vol. 10(2), pp. 130-137, 1992). In this publication, Tsuchiya describes a hyperammonemic patient receiving a combination of carbamazepine, zonisamide, valproic acid, phenytoin, and other drugs. Carbamazepine was discontinued first, followed by valproic acid. No improvement was observed. So the dosage of phenytoin was withdrawn while zonisamide dosage was permitted to continue. Tsuchiya reported that the patient improved following the cessation of phenytoin.

However, a careful review of the data generated in American clinical trials, as well as in AIDR reports gathered once commercial marketing began, has yielded the discovery that zonisamide may independently induce hyperammonemia in a small number of patients, and has implicated hyperammonemia in patients receiving zonisamide as an adjunctive therapy. Accordingly, the present invention is directed to methods of increasing the safety of zonisamide therapy in view of its newly discovered role in hyperammonemia.

Hyperammonemia involves elevated blood ammonia levels that cause a constellation of symptoms that may be characterized as a single disease entity. Normal blood ammonia ranges from 10-40 μmol/L compared to a blood urea nitrogen (BUN) of 6-20 mg/dL. The total soluble ammonia in a normal adult with 5 L of blood circulating is only 150 mcg, in contrast to approximately 1000 mg of urea nitrogen present. Since urea is the end product of ammonia metabolism, the disparity in blood quantities of the substrate and product demonstrate that the metabolic conversion system leading to production of urea is highly efficient. The elimination of ammonia is critical to protecting bodily systems, in particular the central nervous system, from the toxic effects of free ammonia.

It is unlikely that an individual will become hyperammonemic unless the conversion system is impaired in some way. In newborn infants, this impairment often is the result of genetic defects, whereas in older individuals it more often is the consequence of a diseased liver. However, a growing number of reports exist of adult-onset genetic disorders of the urea cycle in previously healthy individuals.

Pathophysiology

The mechanism of neurotoxicity in hyperammonemia is not yet fully determined. Irrespective of the underlying cause, the clinical picture is relatively constant. This implies that the pathophysiologic mechanism, focusing on the CNS, is one that is generally common to all individuals with hyperammonemia.

The normal process of removing the amino group present on all amino acids produces ammonia. The α-amino group is a catabolic key that, when present, keeps amino acids safely locked away from oxidative breakdown. Removing the α-amino group is essential for producing energy from any amino acid. Under normal circumstances, both the liver and the brain generate ammonia in this removal process, contributing substantially to total body ammonia production. The urea cycle is completed in the liver, where urea is generated from free ammonia.

The hepatic urea cycle is the major route for disposal of waste nitrogen generated chiefly from protein and amino acid metabolism. In the same context, low-level synthesis of certain cycle intermediates in extrahepatic tissues makes a small contribution to waste nitrogen disposal as well. Two moles of waste nitrogen are eliminated with each mole of urea excreted. A portion of the cycle is mitochondrial in nature; thus, mitochondrial dysfunction may impair urea production and result in hyperammonemia. Overall, activity of the cycle is regulated by the rate of synthesis of N-acetylglutamate, the enzyme activator that initiates incorporation of ammonia into the cycle.

The brain must expend energy to detoxify and export the ammonia it produces. This is accomplished in the process of producing adenosine diphosphate (ADP) from adenosine triphosphate (ATP) by the enzyme glutamine synthetase, which is responsible for mediating the formation of glutamine from an amino group. Synthesis of glutamine also reduces the total free ammonia circulating in the blood; therefore, a significant increase in blood glutamine concentration can signal hyperammonemia.

Normally, the capacity of the hepatic urea cycle exceeds the normal rates of ammonia generation in the periphery and transfer into the blood. So an elevated blood ammonia level, although it may be secondary, should not be ignored. Moreover, since the normal ureagenic capacity of the liver is so great in relation to physiologic load, such a finding points directly to an impairment of the urea cycle in the liver.

The CNS is most sensitive to the toxic effects of ammonia. Many metabolic derangements occur as a consequence of high ammonia levels, including alteration of metabolism of important compounds such as pyruvate, lactate, glycogen, and glucose. High ammonia also induces changes in N-methyl-D-aspartate (NMDA) and gamma-aminobutyric acid (GABA) receptors, and it causes down-regulation in astroglial glutamate transporter molecules. As ammonia exceeds a normal concentration, an increased disturbance of neurotransmission and synthesis of both GABA and glutamine occurs in the CNS. However, the true. mechanism for neurotoxicity of ammonia is not yet completely defined. The pathophysiology of hyperammonemia is primarily that of a CNS toxin that causes irritability, somnolence, vomiting, cerebral edema, and coma (altered consciousness) leading to death. In some cases, however, a patient's plasma ammonia levels may be elevated without causing abnormal mental status in that individual.

Progressive hyperammonemia, treated or not, eventually causes cerebral edema, coma, and death. Neurological symptoms include poor coordination, dysdiadochokinesia, hypotonia or hypertonia, ataxia, tremor, seizures, lethargy progressing to combativeness to obtundation to coma, and decorticate or decerebrate posturing. While the vast majority of morbidity associated with hyperammonemia derives from the primary cause, repeated hyperammonemic episodes also can cause morbidity. The result, given the direct toxicity of ammonia on the CNS, is a progressive decrease in intellectual function.

Diagnosis

Several laboratory tests are available to diagnose hyperammonemia. The first and most direct test employed is the determination of plasma ammonia levels. This test should be ordered anytime a patient present symptoms suggestive of hyperammonemia. Secondary diagnositc tools include liver function studies (i.e., serum transaminases, prothrombin time/activated partial thromboplastin time (PT/aPTT), alkaline phosphatase, and bilirubin). Abnormal liver function is suggestive of hyperammonemia because severe liver disease is one cause hyperammonemia. However, elevated levels of plasma ammonia can occur even in the absence of any detectable liver dysfunction. Thus, it is important to check plasma ammonia levels even if other tests, such as liver functions, are otherwise normal. Additional secondary diagnostic tests include: plasma amino acid quantitation; urinary organic acid profile (e.g., looking for abnormal increases in propionic acid, methylmalonic acid, isovaleric acid, or other organic acids); urine amino acid levels; and blood gas levels. Skilled medical practitioners are familiar with these tests, as well as others, that may be used to diagnose (or rule out) hyperammonemia.

Treatment

If a patient develops hyperammonemia while on zonisamide therapy, the treating physician should search for other causes of hyperammonemia. Should no other obvious causes be identified, zonisamide may be removed, reduced, or alternatively tapered off such that ammonia levels are at an acceptable level, or alternative treatment for the underlying medical condition be initiated as clinically indicated. If another cause for the hyperammonemia is identified, then it may be possible to carefully rechallenge with zonisamide once the symptoms have subsided. If the patient again appears to be developing hyperammonemia or is diagnosed with hyperammonemia, then switching to another AED could be warranted.

In patients experiencing symptoms of hyperammonemia, the patient's ammonia levels should be estimated promptly, and other appropriate diagnositic tests should be employed as indicated; if these levels are elevated and no other cause is obvious, then the drug should typically be withdrawn or titrated down to a level where the symptoms are no longer prevelant. The patient's ammonia levels should be monitored, as needed, as such symptoms persist, subside, or reoccur.

In patients taking zonisamide who are also being treated with valproic acid and/or carbamazepine, and who manifest the clinical signs and symptoms of hyperammonemia, serum ammonia levels should typically be monitored. If the symptoms of hyperammonemia become significant, then the treating physician should consider reducing, ceasing, or tapering off the zonisamide dosing, while continuing to monitor and assess serum ammonia levels.

In some cases, it may be possible to reduce or taper off the level of zonisamide to avoid hyperammonemia or other side-effects, while maintaining the therapeutic efficacy of the drug therapy. Such decisions may be made by an attending medical personnel, for example, after considering the severity of the hyperammonemia or other side effects in relation to the patient's need for continued zonisamide therapy.

Conventional support measures for mild or severe hyperammonemia are known to skilled medical professionals. In many instances, hyperammonemia is reversible, and plasma ammonia levels return to normal ranges over several months, once the cause has been identified and: addressed. Also, complications from the treatment of the hyperammonemia or its symptoms can be addressed as they arise. For example, abruptly removing anti-epileptic drug therapy from an epileptic patient may result in more severe or more frequent seizures or status epilepticus. Therefore, removal of zonisamide therapy carries the risk of more severe seizures. However, a hospital physician or emergency medical personnel will have access to other pharmacological interventions for short-term control of generalized seizure activity such as either intravenous lorazepam, at a dose of 0.1 mg/kg, or diazepam at 0.2 mg/kg. If sedatives prove insufficient, then a patient also may be administered fosphenytoin, or in status epilepticus, phenobarbital, with careful monitoring for respiratory depression. Intravenous administration is preferred since this route will provide the most rapid attainment of therapeutic serum levels. Additionally, at the treating physician's discretion, an alternate AED may be substituted for zonisamide.

Prevalence in Zonisamide Treated Patients:

The pharmacovigilance data that were collected, reviewed and analyzed provided the following information in respect of the incidence of hyperammonemia. A total of 4 cases fulfilled the criteria of potential hyperammonemia cases. These cases were reviewed in detail for evaluation of possible safety signals. Two US cases were reported as either high ammonia levels or increased ammonia. Two Japanese cases were reported as hyperammonemia.

Of these, two (2) cases (both ZON1000262 and ZON1000346) had strong confounding factors and seem to-be unrelated to zonisamide, but the possibility of zonisamide involvement cannot be completely excluded. The two cases of hyperammonemia originated from Japan and both involved adult patients. Both cases were serious and described as life-threatening. One patient recovered and the outcome of the other case is unknown.

The time course of the development of hyperammonemia occurred about 2.5 months after the initiation of zonisamide in one case and 1 year later in the remaining case. Case (ZON1000363) contained moderate confounding factors, and zonisamide involvement seems unlikely, but cannot be completely excluded. Case (ZON1000533) possessed no confounding obvious factors, and zonisamide involvement is possible. There were no cases of hyperammonemia or hyperammonemia from US sources with no or only weak confounding factors reported.

Estimates of exposure, based upon retail and mail order prescriptions, indicate that the number of unique patients taking zonisamide capsules in the U.S. is about 37,276 (total prescriptions per year/average number of prescriptions per patient per year less a calculated percentage decrease based on estimated annual dropouts) in the time between approval in 2000 and December 2002. Hospital patient data for that period, however, is not-available and is not reflected in the estimates. Estimates of patient exposure for Japan indicate that the number of unique patients taking zonisamide is about 1,185,177 for time beginning with the approval in Japan through-December 2002. Japanese data includes prescription and hospital patient data. Exposure from clinical trials are not included in the U.S. or Japanese exposure estimates. Based on these statistics, the estimated number of patients exposed to zonisamide in the U.S. and Japan is 1,222,453 unique patients. This is a rather conservative estimate, assuring that the number of patients actually exposed to zonisamide is unlikely to be higher than the estimate provided. Similarly, the incidences of NMS estimated herein are unlikely to be higher than calculated. Based on the exposure data and number of hyperammonemia cases, the incidence of hyperammonemia is calculated to be about 1:1,222,453.

The following examples are provided to support the practice of the present invention and are not meant and should not be construed to limit the scope of the claims appended hereto.

### EXAMPLE 1

An 11-year old female patient experienced hepatic dysfunction, lethargy, hypercholesterolemia, increased triglycerides, increased ammonia levels, and increased liver function tests (LFT's) during the use of Zonegran™ for the treatment of Lennox-Gastaut Syndrome. Zonegran™ was started in April 2000 (200 mg, 3 times per day). Prior to administration of the drug, the patient's cholesterol and LFTs were within normal ranges. In December 2000, the patient presented with symptoms of lethargy. On admission to the hospital, the patient was found to have increased cholesterol (735 mg/dL), triglycerides (331 mg/dL), ammonia (140 mcg/dL), GGTP, AST, and ALT. The attending-medical professionals suspected and reported Zonegran™ as the cause, and elected to discontinue the therapy during the patient's hospital admission. The patient was later discharged and events remained ongoing.

### EXAMPLE 2

A 27-year old male patient experienced increased levels of ammonia during the use of Zonegran™ Zonegran was started in July 2000 at a dose of 100 mg daily. The dose was increased every two weeks by 100 mg to a final dose of 400 mg daily. In August and September 2000, the patient was found to have elevated ammonia levels of 165 and 161 ug/dL, respectively (normal values for the reporting laboratory were 27-102 ug/dL). The attending medical professionals suspected and reported Zonegran™ as the cause of the elevated ammonia levels.

### EXAMPLE 3

A 54-year old male patient visited a hospital complaining of clouded consciousness. Ammonia levels were 180 mg/dL. Hepatic disorder was not observed; CT-scan, head MRI, and EEG were normal. Attending medical professionals reported that the clouded consciousness was likely caused by hyperammonemia. Ammonia levels decreased after the patient was withdrawn from Excegran (zonisamide) and Tegretol. After one week, ammonia levels were 64. Normal ammonia levels were maintained and the patient was discharged about one week later.

While this invention has been described with respect to various specific examples and embodiments, it is to be understood that the invention is not limited thereby and should only be construed by interpretation of the scope of the appended claims.

1. A method of using zonisamide as an adjunctive therapy for partial seizures to improve the safety of such therapy comprising:
providing a patient with a therapeutically effective amount of zonisamide, and
informing the patient or the patient's guardian during the course of zonisamide therapy that irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing are symptoms of hyperammonemia that require prompt medical evaluation if such symptoms are experienced by the patient.

2. The method of claim 1, wherein the therapeutically effective amount of zonisamide is from 25 mg to 600 mg.

3. The method of claim 1, wherein the therapeutically effective amount of zonisamide is provided in unit dose form.

4. The method of claim 1, wherein the therapeutically effective amount of zonisamide is provided in a unit dose form and in multiple doses to provide for a course of therapy.

5. The method of claim 4, wherein the unit dose is from 25 mg to 200 mg.

6. A method of using zonisamide as an adjunctive therapy for partial seizures to improve the health of a patient receiving such therapy comprising:
providing a patient with a therapeutically effective amount of zonisamide, and
informing the patient or the patient's guardian during the course of such therapy that irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing are symptoms of hyperammonemia that require prompt medical evaluation if such symptoms are experienced by the patient.

7. The method of claim 6, wherein the therapeutically effective amount of zonisamide is from 25 mg to 600 mg.

8. The method of claim 7, wherein the therapeutically effective amount of zonisamide is provided in unit dose form.

9. The method of claim 6, wherein the therapeutically effective amount of zonisamide is provided in a unit dose form and in multiple doses to provide for a course of therapy.

10. The method of claim 9, wherein the unit dose is from 25 mg to 200 mg.

11. A method of using zonisamide as an adjunctive therapy for partial seizures to reduce the risk of hyperammonemia in a patient receiving such therapy comprising:
providing the patient with a therapeutically effective amount of zonisamide, and
informing the patient or the patient's guardian during the course of zonisamide therapy that irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing are symptoms of hyperammonemia that require prompt medical evaluation if such symptoms are experienced by the patient.

12. The method of claim 11, wherein the therapeutically effective amount of zonisamide is from 25 mg to 600 mg.

13. The method of claim 12, wherein the therapeutically effective amount of zonisamide is provided in unit dose form.

14. The method of claim 11, wherein the therapeutically effective amount of zonisamide is provided in a unit dose form and in multiple doses to provide for a course of therapy.

15. The method of claim 14, wherein the unit dose is from 25 mg to 200 mg.

16. A method of using zonisamide as an adjunctive therapy for partial seizures comprising:
enhancing the safety profile of zonisamide by informing a prescribing physician that hyperammonemia may result from zonisamide therapy and to monitor a patient who is prescribed zonisamide as an adjunctive therapy for irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing;
recommending that, when irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing is observed, an appropriate diagnostic be employed by the physician to determine whether hyperammonemia is present; and
recommending that the physician remove, taper off, or reduce zonisamide dosing in the patient and initiate appropriate supportive therapy.

17. The method of claim 16, wherein the diagnostic comprises measurement of plasma ammonia levels.

18. The method of claim 16, wherein the diagnostic comprises a measurement of a liver enzyme function.

19. A method of using zonisamide as an adjunctive therapy for partial seizures comprising:
improving patient outcome by informing an emergency medical worker that a patient who is receiving zonisamide as an adjunctive therapy for partial seizures and exhibits irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing may be suffering from hyperammonemia; and.
recommending performance of an appropriate diagnostic to determine whether hyperammonemia is present,
and if hyperammonemia is present, recommending that the worker initiate appropriate supportive therapy and discontinue zonisamide dosing in the patient.

20. The method of claim 19, wherein the diagnostic comprises measurement of plasma ammonia levels.

21. The method of claim 19, wherein the diagnostic comprises a measurement of a liver enzyme function.

22. A method of using zonisamide as an adjunctive therapy for partial seizures comprising:
providing packaging that includes a pharmaceutical formulation of zonisamide along with information providing a warning that zonisamide may cause hyperammonemia in some patients and that one or more symptoms chosen from the group of irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia,.extensor plantar signs, and decorticate or decerebrate posturing are potentially signs of hyperammonemia; and
providing the packaging to a patient who has been prescribed zonisamide.

23. A method of using zonisamide as an adjunctive therapy for partial seizures comprising:
enhancing the safety of zonisamide by packaging a pharmaceutical formulation of zonisamide along with information providing a warning that zonisamide may cause hyperammonemia in some patients and that one or more symptoms chosen from the group of irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor, plantar signs, and decorticate or decerebrate posturingare potentially signs of hyperammonemia and providing such packaging to a patient who has been prescribed zonisamide therapy.

24. A method of using zonisamide as an adjunctive therapy for partial seizures comprising:
administering a therapeutically effective amount of zonisamide to a subject in need of treatment;
observing the subject for the appearance of at least one symptom of hyperammonemia; and
if at least one symptom of hyperammonemia is observed in the presence of hyperammonemia, reducing the dosage of the zonisamide to a dosage that does not produce the at least one symptom of hyperammonemia.

25. The method of claim 24, wherein if at least one symptom of hyperammonemia is observed, administration of zonisamide is ceased.

26. The method of claim 24, wherein if at least one symptom of hyperammonemia is observed, the patient is tested for hyperammonemia.

27. The method of claim 26, wherein the testing comprises at least one measurement of plasma ammonia levels or a liver enzyme function.

28. The method of claim 25, further comprising administering a therapeutically effective amount of zonisamide after at least one symptom of hyperammonemia has subsided.

29. The method of claim 24, wherein the therapeutically effective amount of zonisamide is from 25 mg to 600 mg.

30. The method of claim 25, wherein the therapeutically effective amount of zonisamide is provided in unit dose form.

31. The method of claim 30, wherein the therapeutically effective amount of zonisamide is provided in a unit dose form and in multiple doses to provide for a course of therapy.

29. A method of administering zonisamide as an adjunctive therapy for partial seizures comprising:
providing a patient with a therapeutically effective amount of zonisamide and a therapeutically effective amount of at least one other anti-epilepsy drug; and
informing the patient or the patient's guardian that irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing are potentially signs of hyperammonemia that require prompt medical evaluation if such symptoms are experienced by the patient.

30. The method of claim 29, wherein the patient is informed by reference to a package drug insert.

31. The method of claim 30, wherein the patient's guardian is informed by reference to a package drug insert.

32. A method of using zonisamide as an adjunctive therapy for partial seizures comprising:
advising a physician prescribing zonisamide to a patient to monitor the patient for one or more symptoms chosen from the group of irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, and decorticate or decerebrate posturing,
recommending that when irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, or decorticate or decerebrate posturing is observed, an appropriate diagnostic be employed by the physician to determine whether hyperammonemia is present; and
recommending that the physician remove, taper off, or reduce zonisamide dosing in the patient and initiate appropriate supportive therapy.

33. A method for using zonisamide as an adjunctive therapy for partial seizures prescribed by a physician comprising:
monitoring a patient who is receiving administrations of zonisamide for one or more symptoms chosen from the group of irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, and decorticate or decerebrate posturing;
if one or more of said symptoms are observed, determining whether hyperammonemia is present in the patient; and
if hyperammonemia is diagnosed, reducing the zonisamide dosing until the patient's symptoms have subsided.

34. The method of claim 33, wherein the zonisamide dosing is increased after the patient's symptoms have subsided.

35. A method of using zonisamide as an adjunctive therapy for partial seizures prescribed by a physician comprising:
monitoring a patient who is receiving administrations of zonisamide for one or more symptoms chosen from the group of irritability, somnolence, vomiting, cerebral edema, poor coordination, dysdiadochokinesia, hypotonia or hypertonia, ataxia, tremor, seizures, lethargy progressing to combativeness to obtundation to coma, asterixis, rigidity, hyperreflexia, extensor plantar signs, and decorticate or decerebrate posturing;
if one or more of said symptoms are observed, determining whether hyperammonemia is present in the patient; and
if hyperammonemia is diagnosed, ceasing the zonisamide dosing until the symptoms of hyperammonemia have subsided.

36. The method of claim 35, wherein the zonisamide dosing-is restored after the patient's symptoms have subsided.
